(19) United States
(12) Patent Application Publication
(10) Pub. No.: US 2011/0206753 A1
(43) Pub. Date: Aug. 25, 2011

(54) NANOPARTICULATE COPPER COMPOUNDS

(76) Inventors: Andrey Karpov, Mannheim (DE); Hartmut Hibst, Schriesheim (DE); Michael Triller, Ilvesheim (DE); Eike Hupe, Mannheim (DE); Michael Maier, Marxzell (DE); Jörg Habicht, Sinzheim (DE)

(21) Appl. No.: 13/002,812

(22) PCT Filed: Jul. 2, 2009
(86) PCT No.: PCT/EP09/58303

§ 371 Date: May 3, 2011

(30) Foreign Application Priority Data
Jul. 8, 2008 (EP) 08159913.6

(51) Int. Cl. A01N 25/34; A01N 55/02; B01J 31/06; A01P 1/00; C08G 63/91; B82Y 5/00; B82Y 30/00; B82Y 40/00

(52) U.S. Cl. 424/405; 424/78.17; 502/159; 525/418; 977/773; 977/810; 977/896; 977/902

(57) ABSTRACT

The present invention relates to processes for the preparation of surface-modified nanoparticulate copper compounds and of aqueous suspensions which comprise surface-modified nanoparticulate copper compounds. The invention furthermore relates to the surface-modified nanoparticulate copper compounds obtainable by these processes and aqueous suspensions of these copper compounds and their use as an antimicrobial active substance or catalyst.

The present invention relates to processes for the preparation of surface-modified nanoparticulate copper compounds and of aqueous suspensions which comprise surface-modified nanoparticulate copper compounds. The invention furthermore relates to the surface-modified nanoparticulate copper compounds obtainable by these processes and aqueous suspensions of these copper compounds and their use as an antimicrobial active substance or catalyst.

Wood preservatives frequently comprise antimicrobial active substances based on finely divided copper compounds. Thus, WO 2004/091875 describes the use of an aqueous suspension comprising microparticulate copper compounds (e.g. copper hydroxide, copper(I) oxide, copper(II)oxide, copper carbonate) for wood preservation applications. The suspensions were prepared by wet-milling of coarsely crystalline powders in the presence of suitable dispersants and comprise particles having a particle size in the range from 40 nm to 1500 nm at a mean particle size of about 200 nm.

WO 2005/110692 describes aqueous suspensions comprising microparticulate copper compounds (e.g. copper hydroxide, copper carbonate) for wood preservation. The suspensions having mean particle sizes in the range from about 200 nm to about 400 nm were likewise prepared by wet-milling in the presence of dispersants.

The wood preservative preparations disclosed in WO 2006/042128 comprise, inter alia, sparingly soluble copper compounds which were likewise brought into a finely divided form by milling.

The disadvantage of milling processes is that particles having a mean particle size of <100 nm are obtainable only with very great effort by means of a very high energy input.

Over and above milling processes, further processes for the preparation of finely divided copper compounds are known.

Thus US 2002/0112407 describes the preparation of inorganic nanoparticulate particles having a mean size of from 2 to 500 nm, preferably <100 nm (determined by dynamic light scattering, DLS) by partial or complete alkaline hydrolysis of at least one metal compound, which is either dissolved in an aqueous medium or suspended in nanoparticulate form, in the presence of water-soluble comb polymers. A disadvantage of this process is that metal oxides, hydroxides or oxides/hydroxides are always at least partly obtained and hence hydroxide/oxide-free metal compounds are not accessible.

Nanoparticles are defined as particles of the order of magnitude of nanometers. Their size is in the transition region between atomic or monomolecular systems and continuous macroscopic structures. In addition to their mostly very large surface area, nanoparticles are distinguished by particular physical and chemical properties, which differ substantially from those of larger particles. Thus, nanoparticles often have a lower melting point, absorb light only at shorter wavelengths and have mechanical, electrical and magnetic properties differing from those of macroscopic particles of the same material. By using nanoparticles as building blocks, many of these particular properties can also be used for macroscopic materials (Winnacker/Küchler, Chemische Technik: Prozesse and Produkte, (editors: R. Dittmayer, W. Keim, G. Kreysa, A. Oberholz), vol. 2: Neue Technologien, chapter 9, Wiley-VCH Verlag 2004).

In the context of the present invention, the term “nanoparticles” designates particles having a mean diameter of from 1 to 500 nm, determined by means of light scattering.

An object of the present invention was to provide processes for the preparation of surface-modified nanoparticulate copper compounds and of aqueous suspensions which comprise surface-modified nanoparticulate copper compounds. A further object of the invention was to provide novel surface-modified nanoparticulate copper compounds and aqueous suspensions of these copper compounds and their use as an antimicrobial active substance or catalyst.

The invention therefore relates to a process for the preparation of surface-modified nanoparticulate copper compounds, comprising the steps:

a) preparation of an aqueous solution comprising copper ions (solution 1) and of an aqueous solution comprising at least one anion which forms a precipitate with copper ions and is not a hydroxide ion (solution 2), at least one of the two solutions 1 and 2 comprising at least one water-soluble polymer,

b) mixing of the solutions 1 and 2 prepared in step a), at a temperature in the range from 0 to 100° C., with the surface-modified nanoparticulate copper compounds forming and being precipitated from the solution with formation of an aqueous dispersion,

c) isolation of the surface-modified nanoparticulate copper compounds from the aqueous dispersion obtained in step b), and

d) if appropriate, drying of the surface-modified nanoparticulate copper compounds obtained in step c).

The copper compounds obtainable by the process according to the invention may be present both in anhydrous form and in the form of corresponding hydrates.

The preparation of the solution 1 described in step a) can be effected, for example, by dissolving a water-soluble copper salt in water or an aqueous solvent mixture. An aqueous solvent mixture may also comprise, for example, water-miscible alcohols, ketones or esters, such as methanol, ethanol, acetone or ethyl acetate, in addition to water. The water content in such a solvent mixture is usually at least 50% by weight, preferably at least 80% by weight.

The water-soluble copper salts may be, for example, copper(II) halides, acetates, sulfates or nitrates. Preferred copper salts are copper chloride, copper acetate, copper sulfate and copper nitrate. These salts dissolve in water with formation of copper ions which have a double positive charge and to which six water molecules are attached [Cu(H₂O)₆²⁺].

The concentration of the copper ions in the solution 1 is as a rule in the range from 0.05 to 2 mol/l, preferably in the range from 0.1 to 1 mol/l.

In addition to the copper ions, solution 1 may also comprise further metal ions (M^{k+}) which, if appropriate, are precipitated in step b) together with the copper ions. Said further metal ions may be, for example, ions of alkaline earth metals or transition metals, preferably magnesium, calcium, chromium, cobalt, nickel, zinc or silver ions, particularly preferably zinc or silver ions. The other metal ions are present here in a smaller number than the copper ions.

In the process according to the invention, solution 2 comprises at least one anion which forms a precipitate with copper ions and is not a hydroxide ion, This anion is, for example, an anion of mineral acids, such as hydrochloric acid, sulfuric acid, phosphoric acid, carbonic acid, boric acid, sulfurous acid, etc., or an anion of organic acids, such as oxalic acid, benzoic acid, maleic acid, etc., and a polyborate such as B₄O₇²⁻. In addition, solution 2 can of course additionally comprise hydroxide ions.

In a further embodiment of the invention, the anion which forms a precipitate with copper ions and is not a hydroxide ion may be formed from a precursor compound only in the course of the reaction taking place in step b). Here, the anion is present in the precursor compound in masked form and is liberated from it on mixing of the solutions 1 and 2 and/or by a change in temperature. The precursor compound may be present either in solution 1 or in solution 2 or in both solutions. Dimethyl carbonate, from which carbonate ions can be liberated in an alkaline medium, may be mentioned as an example of such a precursor compound (cf. M. Faatz et al., Adv. Mater. 2004, vol. 16, pages 996 to 1000).

According to the invention, at least one of the two solutions 1 and 2 comprises at least one water-soluble polymer. In the context of this invention, a “water-soluble polymer” is understood as meaning a polymer of which in general at least 0.01% by weight dissolves in water at room temperature and which forms a clear single-phase solution without turbidity up to a concentration of 50% by weight in water, preferably 75% by weight in water. The at least one water-soluble polymer serves for surface modification of the copper compounds and helps to stabilize them in nanoparticulate form.

The water-soluble polymers to be used according to the invention may be anionic, cationic, nonionic or zwitterionic polymers. Their molecular weight is in general in the range from about 800 to about 500 000 g/mol, preferably in the range from about 1000 to about 30 000 g/mol. They may be homo- or copolymers and their molecular structure may be either linear or branched. Water-soluble polymers having a comb structure are preferred.

Suitable monomers from which the water-soluble polymers to be used according to the invention are obtainable comprise, for example, α,β-unsaturated carboxylic acids and esters, amides and nitriles thereof, N-vinylcarboxamides, alkylene oxides, unsaturated sulfonic acids and phosphonic acids and amino acids.

In an embodiment of the invention, polycarboxylates are used as water-soluble polymers. In this invention, polycarboxylates are polymers based on at least one α,β-unsaturated carboxylic acid, for example acrylic acid, methacrylic acid, dimethacrylic acid, ethacrylic acid, maleic acid, citraconic acid, methylenemalonic acid, crotonic acid, isocrotonic acid, fumaric acid, mesaconic acid and itaconic acid. Polycarboxylates based on acrylic acid, methacrylic acid, maleic acid or mixtures thereof are preferably used.

The proportion of the at least one α,β-unsaturated carboxylic acid in the polycarboxylates is as a rule in the range from 20 to 100 mol %, preferably in the range from 50 to 100 mol %, particularly preferably in the range from 75 to 100 mol %.

The polycarboxylates to be used according to the invention can be used both in the form of the free acid and partly or completely neutralized in the form of their alkali metal, alkaline earth metal or ammonium salts. However, they can also be used as salts of the respective polycarboxylic acid and triethylamine, ethanolamine, diethanolamine, triethanolamine, morpholine, diethylenetriamine or tetraethylenepentamine.

In addition to the at least one α,β-unsaturated carboxylic acid, the polycarboxylates may comprise further comonomers which are incorporated in the form of polymerized units in the polymer chain, for example the esters, amides and nitriles of the abovementioned carboxylic acids, such as methyl acrylate, ethyl acrylate, methyl methacrylate, ethyl methacrylate, hydroxyethyl acrylate, hydroxypropyl acrylate, hydroxybutyl acrylate, hydroxyethyl methacrylate, hydroxypropyl methacrylate, hydroxyisobutyl acrylate, hydroxyisobutyl methacrylate, methyl maleate, dimethyl maleate, monoethyl maleate, diethyl maleate, 2-ethylhexyl acrylate, 2-ethylhexyl methacrylate, acrylamide, methacrylamide, N-dimethylacrylamide, N-tert-butylacrylamide, acrylonitrile, methacrylonitrile, dimethylaminoethyl acrylate, diethylaminoethyl acrylate, diethylaminoethyl methacrylate and the salts of the last-mentioned basic monomers with carboxylic acids or mineral acids and the quaternized products of the basic (meth)acrylates.

Allylacetic acid, vinylacetic acid, acrylamidoglycolic acid, vinylsulfonic acid, allylsulfonic acid, methallylsulfonic acid, styrenesulfonic acid, 3-sulfopropyl acrylate, 3-sulfopropyl methacrylate or acrylamidomethylpropanesulfonic acid and monomers comprising phosphonic acid groups, such as vinylphosphonic acid, allylphosphonic acid or acrylamidomethylpropanephosphonic acid, are also suitable as further comonomer which can be incorporated in the form of polymerized units. The monomers comprising acid groups can be used in the polymerization in the form of the free acid groups and in a form partly or completely neutralized with bases.

Further suitable copolymerizable compounds are N-vinylcaprolactam, N-vinylimidazole, N-vinyl-2-methylimidazole, N-vinyl-4-methylimidazole, vinyl acetate, vinyl propionate, isobutene, styrene, ethylene oxide, propylene oxide or ethyleneimine and compounds having more than one polymerizable double bond, such as, for example, diallylammonium chloride, ethylene glycol dimethacrylate, diethylene glycol diacrylate, allyl methacrylate, trimethylolpropane triacrylate, triallylamine, tetraallyloxyethane, triallyl cyanurate, diallyl maleate, tetraallylethylenediamine, divinylideneurea, pentaerythrityl diallyl ether, pentaerythrityl triallyl ether and pentaerythrityl tetraallyl ether, N,N″-methylenebisacrylamide or N,N″-methylenebismethacrylamide.

It is of course also possible to use mixtures of said comonomers. For example, mixtures of from 50 to 100 mol % of acrylic acid and from 0 to 50 mol % of one or more of said comonomers are suitable for the preparation of the polycarboxylates according to the invention.

In a preferred embodiment of the invention, polycarboxylate ethers are used as water-soluble polymers.

Numerous polycarboxylates to be used according to the invention are commercially available under the trade name Sokalan® (from BASF SE).

In further embodiments of the invention, the water-soluble polymer is polyaspartic acid, polyvinylpyrrolidone or a copolymer of N-vinylamide, for example N-vinylpyrrolidone, and at least one further monomer comprising a polymerizable group, for example with monoethylenically unsaturated C₃-C₈-carboxylic acids, such as acrylic acid, methacrylic acid, C₈-C₃₀-alkyl esters of monoethylenically unsaturated C₃-C₈-carboxylic acids, vinyl esters of aliphatic C₈-C₃₀-carboxylic acids and/or N-alkyl- or N,N-dialkyl-substituted amides of acrylic acid or of methacrylic acid having C₈-C₁₈-alkyl radicals.

In a preferred embodiment of the process according to the invention, the water-soluble polymer used is polyaspartic acid. In the context of the present invention, the term polyaspartic acid comprises both the free acid and the salts of polyaspartic acid, e.g. sodium, potassium, lithium, magnesium, calcium, ammonium, alkylammonium, zinc and iron salts or mixtures thereof.

In a further embodiment of the invention, nonionic water-soluble polymers are used. In the context of this invention, a nonionic water-soluble polymer is a surface-active substance whose chemical structure comprises from 2 to 1000 —CH₂CH₂O— groups, preferably from 2 to 200 —CH₂CH₂O— groups, particularly preferably from 2 to 80 —CH₂CH₂O— groups. These groups form, for example, by an addition reaction of a corresponding number of ethylene oxide molecules with substrates containing hydroxyl or carboxyl groups and as a rule form one or more cohesive ethylene glycol chains whose chemical structure corresponds to the formula —(CH₂CH₂O—)ₙ— where n is from about 2 to about 80.

In a preferred embodiment of the invention, the nonionic water-soluble polymer used is at least one substance from one of the following groups:

Adducts of from 2 to 80 mol of ethylene oxide and, if appropriate, from 1 to 15 mol of propylene oxide with
alkylphenols having 1 to 5 carbon atoms in the alkyl group,
glycerol mono- and diesters, sorbitol mono- and diesters and sorbitan mono- and diesters of saturated and unsaturated fatty acids having 6 to 22 carbon atoms,
alkylmono- and -oligoglycosides having 1 to 5 carbon atoms in the alkyl radical,
acetic acid,
lactic acid,
glycerol,
polyglycerol,
pentaerythritol,
dipentaerythritol,
sucrose,
sugar alcohols (e.g. sorbitol),
alkylglucosides (e.g. methylglucoside, butylglucoside, laurylglucoside),

polyglucosides (e.g. cellulose).

Polyalkylene glycols whose structure comprises from 2 to 80 ethylene glycol units.

In a particularly preferred embodiment of the invention, the nonionic water-soluble polymer used is at least one substance from one of the following groups:

Adducts of from 2 to 80 mol of ethylene oxide with
alkylphenols having 1 to 5 carbon atoms in the alkyl group,
glycerol, and
alkylglucosides.

Numerous nonionic water-soluble polymers to be used according to the invention are commercially available under the trade name Cremophor® (from BASF SE).

The ethylene oxide adducts in technical quality may still comprise a small proportion of the substrates listed above by way of example and containing free hydroxyl or carboxyl groups. As a rule, this proportion is less than 20% by weight, preferably less than 5% by weight, based on the total amount of the nonionic water-soluble polymer.

In a further embodiment of the invention, water-soluble polymers used are homo- and copolymers of N-vinylcarboxamides. These polymers are prepared by homo- or copolymerization of, for example, N-vinylformamide, N-vinylacetamide, N-alkyl-N-vinylformamide or N-alkyl-N-vinylacetamide. Among the N-vinylcarboxamides, N-vinylformamide is preferably used, homopolymers of N-vinylformamide being particularly preferred.

The water-soluble N-vinylcarboxamide polymers to be used according to the invention can, if appropriate, also comprise from 0 to 80, preferably from 5 to 30% by weight of comonomers incorporated in the form of polymerized units, in each case based on the total composition of the polymers, in addition to from 100 to 20% by weight of the N-vinylcarboxamides. The comonomers are, for example, monoethylenically unsaturated carboxylic acids having 3 to 8 carbon atoms, such as acrylic acid, methacrylic acid, dimethacrylic acid, ethacrylic acid, maleic acid, citraconic acid, methylenemalonic acid, allylacetic acid, vinylacetic acid, crotonic acid, fumaric acid, mesaconic acid and itaconic acid. From this group of monomers, acrylic acid, methacrylic acid, maleic acid or mixtures of said carboxylic acids are preferably used. The monoethylenically unsaturated carboxylic acids are used in the copolymerization either in the form of the free acids or in the form of their alkali metal, alkaline earth metal or ammonium salts. However, they can also be used as salts of the respective acid and triethylamine, ethanolamine, diethanolamine, triethanolamine, morpholine, diethylenetriamine or tetraethylenepentamine.

Further suitable comonomers are, for example, the esters, amides and nitriles of the abovementioned carboxylic acids, e.g. methyl acrylate, ethyl acrylate, methyl methacrylate, ethyl methacrylate, hydroxyethyl acrylate, hydroxypropyl acrylate, hydroxybutyl acrylate, hydroxyethyl methacrylate, hydroxypropyl methacrylate, hydroxyisobutyl acrylate, hydroxyisobutyl methacrylate, monomethyl maleate, dimethyl maleate, monoethyl maleate, diethyl maleate, 2-ethylhexyl acrylate, 2-ethylhexyl methacrylate, acrylamide, methacrylamide, N-dimethylacrylamide, N-tert-butylacrylamide, acrylonitrile, methacrylonitrile, dimethylaminoethyl acrylate, diethylaminoethyl acrylate, diethylaminoethyl methacrylate and the salts of the last-mentioned basic monomers with carboxylic acids or mineral acids and the quaternized products of the basic (meth)acrylates. Acrylamide or methacrylamide is preferably used.

Acrylamidoglycolic acid, vinylsulfonic acid, allylsulfonic acid, methallylsulfonic acid, styrenesulfonic acid, 3-sulfopropyl acrylate, 3-sulfopropyl methacrylate or acrylamidomethylpropanesulfonic acid and monomers comprising phosphonic acid groups, such as vinylphosphonic acid, allylphosphonic acid or acrylamidomethanepropanephosphonic acid, are also suitable as further comonomers which can be incorporated in the form of polymerized units. The monomers comprising acid groups can be used in the polymerization in the form of the free acid groups and in a form partly or completely neutralized with bases.

Further suitable copolymerizable compounds are N-vinylpyrrolidone, N-vinyl-caprolactam, N-vinylimidazole, N-vinyl-2-methylimidazole, N-vinyl-4-methyl-imidazole, vinyl acetate, vinyl propionate, isobutene, styrene, ethylene oxide, propylene oxide or ethyleneimine and compounds having more than one polymerizable double bond such as, for example, diallylammonium chloride, ethylene glycol dimethacrylate, diethylene glycol diacrylate, allyl methacrylate, trimethylol propane triacrylate, triallylamine, tetraallyloxyethane, triallyl cyanurate, diallyl maleate, tetraallylethylenediamine, divinylideneurea, pentaerythrityl diallyl ether, pentaerythrityl triallyl ether and pentaerythrityl tetraallyl ether, N,N″-methylenebisacrylamide or N,N″-methylenebismethacrylamide.

It is of course also possible to use mixtures of said comonomers. For example, mixtures of from 50 to 100% by weight of N-vinylformamide and from 0 to 50% by weight of one or more of said comonomers are suitable for the preparation of the water-soluble polymers according to the invention.

If said comonomers do not give water-soluble polymers when polymerized alone, the polymers comprising N-vinylcarboxamide units comprise these comonomers incorporated in the form of polymerized units only in amounts such that the copolymers are still water-soluble.

In a preferred embodiment of the invention, nonionic water-soluble polymers having a comb-like molecular structure are used, which polymers are obtained, for example, by copolymerization of monomer mixtures comprising macromonomers. The structure of the nonionic water-soluble polymers having a comb-like molecular structure can be described, for example, as a complex-forming polymer backbone having anionic and/or cationic groups and hydrophilic side chains or as a neutral hydrophilic polymer backbone having complex-forming anionic and/or cationic groups.

In the context of this invention, macromonomers are understood as meaning substances which, at a molecular weight of preferably less than 500 000 D, in particular in the range from 300 to 100 000 D, particularly preferably in the range from 500 to 20 000 D, very particularly preferably in the range from 800 to 15 000 D, have a substantially linear molecular structure and carry a polymerizable terminal group at one end.

In a preferred embodiment of the invention, macromonomers which are based on polyalkylene glycols and are provided with a polymerizable terminal group at one end are used for the preparation of the water-soluble polymers having a comb-like molecular structure. Said polymerizable terminal group may be, for example, a vinyl, allyl, (meth)acrylic acid or (meth)acrylamide group, the corresponding macromonomers being described by the following formulae:

CH₂=CR²—P, (II)

CH₂=CH—CH₂—P, (III)

CH₂=CH—CH₂—NH—R³—P, (IV)

CH₂=CH—CH₂—CO—P, (V)

CH₂=CR²—CO—P, (VI)

CH₂=CR²—CO—NH—R³—P, (VII)

CH₂=CR²—CO—O—R³—P, (VIII)

where

R²=is H or methyl,

R³ is as defined below and

P is a polyalkylene glycol radical of the general formula

P=—{—O—(R³O)ᵤ—R⁴O)ᵥ—(R⁵O)_{w}—[—A—(R⁶O)ₓ—(R⁷O)_{y}—(R⁸O)_{z}—]R⁹}ₙ

in which the variables, independently of one another, have the following meaning:

R⁹ is hydrogen, NH₂, C₁-C₈-alkyl, R¹⁰—C(═O)—, R¹⁰—NH—C(═O)—;

R³ to R⁸ are

—(CH₂)₂—, —(CH₂)₃—, —(CH₂)₄—, —CH₂—CH(CH₃)—, —CH₂—CH(CH₂—CH₃)—, —CH₂—CHOR¹¹—CH₂—;

R¹⁰ is C₁-C₈-alkyl;

R¹¹ is hydrogen, C₁-C₈-alkyl, R¹⁰—C(═O)—;

A is —C(═O)—O—, —C(═O)—B—C(═O)—O—, —C(═O)—NH—B—NH—C(═O)—O—;

B is —(CH₂)ₜ—, arylene, optionally substituted;

n is from 1 to 8;

s is from 0 to 500, preferably from 0 to 20;

t is from 1 to 8;

u is from 1 to 5000, preferably from 1 to 1000, particularly preferably from 1 to 100;

v is from 0 to 5000, preferably from 0 to 1000;

w is from 0 to 5000, preferably from 0 to 1000;

x is from 1 to 5000, preferably from 1 to 1000;

y is from 0 to 5000, preferably from 0 to 1000;

z is from 0 to 5000, preferably from 0 to 1000.

Preferred compounds are those in which the polyalkylene glycol radical P is derived from a polyalkylene glycol which has been prepared using ethylene oxide, propylene oxide and butylene oxide and polytetrahydrofuran. Depending on the type of monomer building blocks used here, the result is a polyalkylene glycol radical P having the following structural units.

—(CH₂)₂—O—, —(CH₂)₃—O—, —(CH₂)₄—O—, —CH₂—CH(CH₃)—O—, —CH₂—CH(CH₂—CH₃)—O—, —CH₂-—CHOR¹¹—CH₂—O—;

The terminal primary hydroxyl group of the polyalkylene glycol radical P (R⁹=H) can be either present in free form or etherified or esterified with alcohols having a chain length of C₁-C₈ or with carboxylic acids having a chain length of C₁-C₈, respectively. However, they can also be exchanged for primary amino groups by reductive amination with hydrogen/ammonia mixtures under pressure or converted into terminal aminopropyl groups by cyanoethylation with acrylonitrile and hydrogenation.

Branched or straight C₁-C₈-alkyl chains, preferably methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, n-heptyl, 2-ethylhexyl and n-octyl, may be mentioned as alkyl radicals R⁹ to R¹¹.

Branched or straight C₁-C₆-alkyl chains, particularly preferably C₁-C₄-alkyl chains, may be mentioned as preferred members of the abovementioned alkyl radicals.

These water-soluble polymers having a comb-like molecular structure also comprise as a rule from about 10 to 90, preferably from 25 to 70, % by weight of comonomers which are incorporated in the form of polymerized units and carry deprotonatable groups, in addition to from about 90 to 10% by weight of the macromonomers described. Comonomers may be, for example, monoethylenically unsaturated carboxylic acids having 3 to 8 carbon atoms, such as acrylic acid, methacrylic acid, dimethacrylic acid, ethacrylic acid, maleic acid, citraconic acid, methylenemalonic acid, allylacetic acid, vinylacetic acid, crotonic acid, fumaric acid, mesaconic acid and itaconic acid. From this group of comonomers, acrylic acid, methacrylic acid, maleic acid or mixtures of said carboxylic acids are preferably used. The monoethylenically unsaturated carboxylic acids are used in the copolymerization either in the form of the free acids or in the form of their alkali metal, alkaline earth metal or ammonium salts. However, they may also be used as salts of the respective acid and triethylamine, ethanolamine, diethanolamine, triethanolamine, morpholine, diethylenetriamine or tetraethylenepentamine.

Further suitable comonomers are, for example, the esters, amides and nitriles of the abovementioned carboxylic acids, e.g. methyl acrylate, ethyl acrylate, methyl methacrylate, ethyl methacrylate, hydroxyethyl acrylate, hydroxypropyl acrylate, hydroxybutyl acrylate, hydroxyethyl methacrylate, hydroxypropyl methacrylate, hydroxyisobutyl acrylate, hydroxyisobutyl methacrylate, monomethyl maleate, dimethyl maleate, monoethyl maleate, diethyl maleate, 2-ethylhexyl acrylate, 2-ethylhexyl methacrylate, acrylamide, methacrylamide, N-dimethylacrylamide, N-tert-butylacrylamide, acrylonitrile or methacrylonitrile, which, after their incorporation in the form of polymerized units into the water-soluble polymers having a comb-like molecular structure, can be hydrolyzed to give the corresponding free carboxylic acids.

It is of course also possible to use mixtures of said comonomers. The monomers may be present in the copolymers in random distribution or as so-called block polymers.

If said comonomers do not give water-soluble polymers when polymerized alone, the water-soluble polymers comprising macromonomers and having a comb-like molecular structure comprise these comonomers incorporated in the form of polymerized units only in amounts such that they are still water-soluble.

The concentration of the water-soluble polymers in the solutions 1 and/or 2 prepared in process step a) is as a rule in the range from 0.1 to 30 g/l, preferably from 1 to 25 g/l, particularly preferably from 5 to 20 g/l.

The mixing of the two solutions 1 and 2 in process step b) is effected at a temperature in the range from 0° C. to 100° C., preferably in the range from 10° C. to 95° C., particularly preferably in the range from 15° C. to 80° C.

The time for mixing the two solutions in process step b) is, for example, in the range from 1 second to 6 hours, preferably in the range from 1 minute to 2 hours. In general, the mixing time in the batchwise procedure is longer than in the continuous procedure.

The mixing in process step b) can be effected, for example, by combining an aqueous solution of a copper salt, for example of copper acetate or copper nitrate, with an aqueous solution of a mixture of a polyacrylate and oxalic acid. Alternatively, an aqueous solution of a mixture of a polyacrylate and a copper salt, for example of copper acetate or copper nitrate, can also be combined with an aqueous oxalic acid solution. Furthermore, an aqueous solution of a mixture of a polyacrylate and a copper salt, for example of copper acetate or copper nitrate, can also be combined with an aqueous solution of a mixture of a polyacrylate and oxalic acid.

In a preferred embodiment of the invention, the mixing in process step b) is effected by metering an aqueous solution of a mixture of a polyacrylate and oxalic acid into an aqueous solution of a mixture of a polyacrylate and a copper salt, for example of copper acetate or copper nitrate, or by metering an aqueous oxalic acid solution into an aqueous solution of a mixture of a polyacrylate and a copper salt, for example of copper acetate or of copper nitrate.

During the mixing or after the mixing, the surface-modified nanoparticulate copper compounds form and are precipitated from the solution with the formation of an aqueous suspension. The mixing is preferably effected with simultaneous stirring of the mixture. After complete combination of the two solutions 1 and 2, the stirring is preferably continued for a further time in the range from 30 minutes to 5 hours at a temperature in the range from 0° C. to 100° C.

In a further preferred embodiment of the process according to the invention, at least one of the process steps a) to d) is carried out continuously. In the continuously operated procedure, process step b) is preferably carried out in a tubular reactor.

The isolation of the precipitated copper compounds from the aqueous suspension in process step c) can be effected in a manner known per se, for example by filtration or centrifuging. If required, the aqueous dispersion can be concentrated before the isolation of the precipitated copper compounds, for example by means of a membrane process, such as nanofiltration, ultrafiltration, microfiltration or crossflow filtration and, if appropriate, at least partly freed from undesired water-soluble constituents, for example alkali metal salts, such as sodium acetate or sodium nitrate.

It has proven advantageous if the isolation of the surface-modified nanoparticulate copper compounds from the aqueous suspension obtained in step b) is carried out at a temperature in the range from 10 to 50° C., preferably at room temperature. It is therefore advantageous to cool the aqueous suspension obtained in step b), if appropriate, to such a temperature.

In process step d), the filtercake obtained can be dried in a manner known per se, for example in a drying oven at temperatures of from 40 to 100° C., preferably from 50 to 80° C., under atmospheric pressure to constant weight.

The surface-modified nanoparticulate copper compounds obtainable by the process according to the invention have as a rule particle sizes in the range from 1 to 200 nm, preferably in the range from 1 to 100 nm.

The present invention furthermore relates to surface-modified nanoparticulate copper compounds having a chemical composition according to the general formula

[Cu²⁺]₁₋ₓ[M^{k+}]ₓ[Xⁿ⁻]ₐ[Y^{m-}]_{b}·e H₂O,

where

M^{k+} is a metal ion having the valency k,

0≦x ≦0.5,

Xⁿ⁻ is an anion having the valency n, which forms a precipitate with copper ions and is not a hydroxide ion,

Y^{m-} is an anion having the valency m,

a>0, b≧0 and the ratio of a, b and x depends on the valencies k, n and m according to the formula a·n+b·m=2·(1-x)+x·k,

e≧0,

having a particle diameter of from 1 to 200 nm, which copper compounds are obtainable by the process described above.

The valencies of the ions are of course integers.

The metal ions M^{k+} may be, for example, ions of alkaline earth metals or transition metals, preferably magnesium, calcium, chromium, cobalt, nickel, zinc or silver ions, particularly preferably zinc or silver ions. The metal ions M^{k+} are present in a smaller number than the copper ions (0≦x≦0.5).

The anions Xⁿ⁻ and Y^{m-} may be, for example, anions of mineral acids, such as hydrochloric acid, sulfuric acid, phosphoric acid, carbonic acid, boric acid, sulfurous acid, etc., or anions of organic acids, such as oxalic acid, benzoic acid, maleic acid, etc., and polyborates such as B₄O₇²⁻. Y^{m-} may also be a hydroxide ion.

In a preferred embodiment of the invention, x is 0. In a further preferred embodiment of the invention, Xⁿ⁻ is selected from the group consisting of carbonate, phosphate, hydrogen phosphate, oxalate, borate and tetraborate ions.

The present invention furthermore relates to the use of surface-modified nanoparticulate copper compounds which are prepared by the process according to the invention as an antimicrobial active substance or catalyst.

According to a preferred embodiment of the present invention, the surface-modified nanoparticulate copper compounds are redispersible in a liquid medium and form stable dispersions. This is particularly advantageous because the dispersions prepared from the copper compounds according to the invention must not be redispersed before further processing but can be processed over a relatively long period.

According to a further preferred embodiment of the present invention, the surface-modified nanoparticulate copper compounds are redispersible in water and form stable dispersions there. Since numerous applications of the surface-modified nanoparticulate copper compounds according to the invention require their use in the form of an aqueous dispersion, their isolation as a solid can, if appropriate, be dispensed with.

The present invention therefore further relates to a process for the preparation of an aqueous dispersion of surface-modified nanoparticulate copper compounds, comprising the steps

a) preparation of an aqueous solution comprising copper ions (solution 1) and of an aqueous solution comprising at least one anion which forms a precipitate with copper ions and is not a hydroxide ion (solution 2), at least one of the two solutions 1 and 2 comprising at least one water-soluble polymer,

b) mixing of the solutions 1 and 2 prepared in step a), at a temperature in the range from 0 to 100° C., with the surface-modified nanoparticulate copper compounds forming and being precipitated from the solution with formation of an aqueous dispersion,

c) if appropriate, concentration of the resulting aqueous dispersion and/or removal of by-products.

Regarding a more detailed description of the procedure of process steps a) and b), of the starting materials and process parameters used and of the product properties, reference is made to the statements further above.

If required, the aqueous dispersion formed in step b) can be concentrated in process step c), for example if a higher solids content is desired. The concentration can be carried out in a manner known per se, for example by distilling off the water (at atmospheric pressure or at reduced pressure), filtering or centrifuging.

It may furthermore be necessary to separate off by-products in process step c) from the aqueous dispersion formed in step b), namely when said by-products would disturb the further use of the dispersion. Suitable by-products are primarily salts which are dissolved in water and form in addition to the desired surface-modified nanoparticulate copper compound during the reaction according to the invention between the solutions 1 and 2, for example sodium chloride, sodium nitrate or ammonium chloride. Such by-products can be substantially removed from the aqueous dispersion, for example, by means of a membrane process, such as nanofiltration, ultrafiltration, microfiltration or crossflow filtration.

In a further preferred embodiment of the present invention, at least one of the process steps a) to c) is carried out continuously.

The present invention furthermore relates to aqueous dispersions of surface-modified nanoparticulate copper compounds having a chemical composition according to the general formula

[Cu²⁺]₁₋ₓ[M^{k+}]ₓ[Xⁿ⁻]ₐ[Y^{m-}]_{b}·e H₂O,

where

M^{k+} is a metal ion having the valency k,

0≦x≦0.5,

Xⁿ⁻ is an anion having the valency n, which forms a precipitate with copper ions and is not a hydroxide ion,

Y^{m-} is an anion having the valency m,

a>0, b≧0 and the ratio of a, b and x depends on the valencies k, n and m according to the formula a·n+b·m=2·(1-x)+x·k,

e≧0,

having a particle diameter of from 1 to 200 nm, which dispersions are obtainable by the process described above.

Regarding a more detailed description of the composition and parameters, of the starting materials and process conditions used and of the product properties, reference is made to the statements further above.

According to a preferred embodiment of the invention, the surface-modified nanoparticulate copper compounds in the aqueous dispersions according to the invention are coated with a polycarboxylate, for example with a polycarboxylate ether.

The present invention furthermore relates to the use of aqueous dispersions of surface-modified nanoparticulate copper compounds which are prepared by the process according to the invention as an antimicrobial active substance or as a catalyst.

The invention is to be explained in more detail with reference to the following examples.

### EXAMPLES

Particle size distributions were measured by light scattering on the Nanotrac U2059I apparatus (from Microtrac Inc. Examples 1 and 2) or on the Zetasizer Nano S apparatus (from Malvern Instruments, Examples 3 and 4). The mean particle size is determined according to the volume fraction.

### Example 1

Batchwise preparation of nanoparticulate copper oxalate in the presence of Sokalan® HP 80 (modified polycarboxylate ether, MW=20 000 g/mol)

Two aqueous solutions 1 and 2 were first prepared. The solution 1 comprised 79.8 g of copper acetate (Sigma-Aldrich, Cu content 32 g/100 g) per liter and had a copper ion concentration of 0.4 mol/l. In addition, the solution 1 comprised 20 g/l of Sokalan® HP 80 (BASF SE, solids content=40% by weight).

The solution 2 comprised 36 g of oxalic acid per liter and thus had an oxalate ion concentration of 0.4 mol/l. In addition, the solution 2 comprised 20 g/l of Sokalan® HP 80 (BASF SE, solids content=40% by weight).

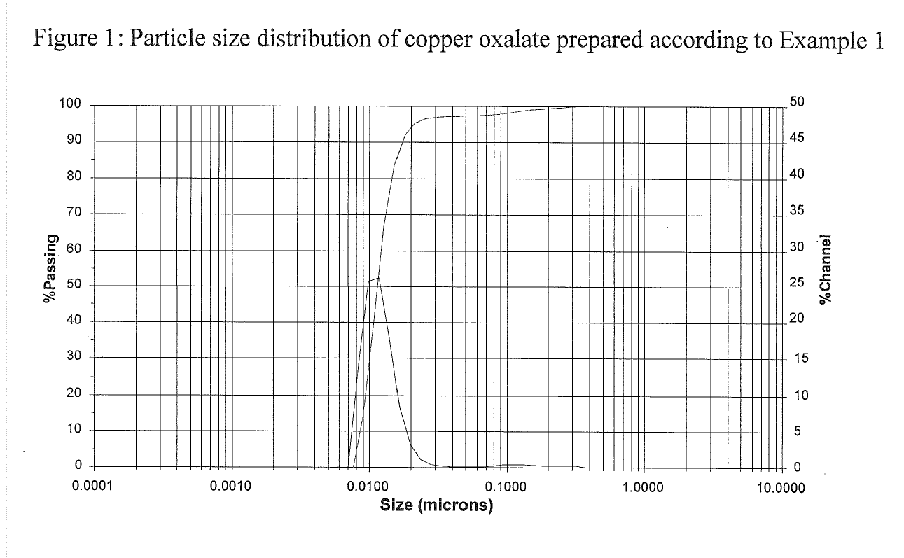

100 ml of the solution 1 were heated to 60° C. 100 ml of the solution 2 were metered into the solution 1 with stirring in the course of 1 minute. The resulting reaction mixture was then stirred for a further 15 minutes. The blue suspension obtained was transferred via a 0.45 μm filter. The filtered suspension had a mean particle size of about 6 nm (FIG. 1).

### Example 2

Batchwise preparation of nanoparticulate copper hydroxocarbonate in the presence of Sokalan® HP 80 (modified polycarboxylate ether, MW=20 000 g/mol)

Two aqueous solutions 1 and 2 were first prepared. The solution 1 was prepared at 75° C. and comprised 139.65 g of copper acetate (Sigma-Aldrich, Cu content 32 g/100 g) per liter and had a copper ion concentration of 0.7 mol/l. In addition, the solution 1 comprised 31.85 g/l of dimethyl carbonate (Acros Organics) and 35 g/l of Sokalan® HP 80 (BASF SE, solids content=40% by weight).

The solution 2 comprised 28 g of sodium hydroxide per liter and thus had a hydroxyl ion concentration of 0.7 mol/l. In addition, the solution 2 comprised 35 g/l of Sokalan® HP 80 (BASF SE, solids content=40% by weight).

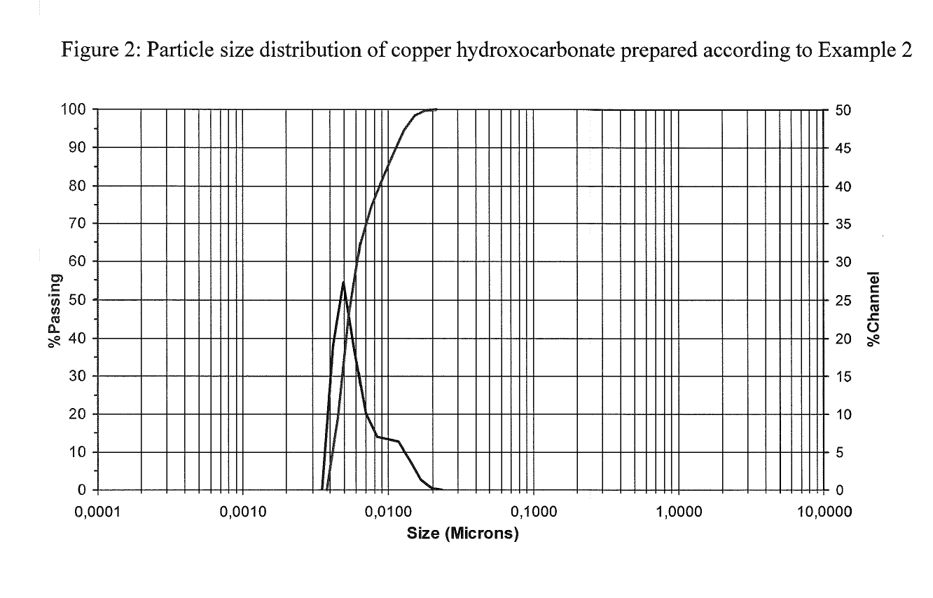

2000 ml of the solution 2 were metered into 2000 ml of the solution 1, which was kept at 75° C., with stirring in the course of 15 minutes. The resulting reaction mixture was then stirred for a further 15 minutes, The green suspension obtained was transferred via a 0.45 μm filter. The filtered suspension had a mean particle size of about 11 nm (FIG. 2).

### Example 3

Batchwise preparation of nanoparticulate copper hydroxocarbonate in the presence of Sokalan® HP 80 (modified polycarboxylate ether, MW=20 000 g/mol)

Two aqueous solutions 1 and 2 were first prepared. The solution 1 was prepared at room temperature and comprised 39.7 g of copper acetate (Sigma-Aldrich, Cu content 32 g/100 g) per liter and had a copper ion concentration of 0.2 mol/l. In addition, the solution 1 comprised 50 g/l of Sokalan® HP 80 (BASF SE, solids content=40% by weight).

The solution 2 comprised 8 g of sodium hydroxide per liter and thus had a hydroxyl ion concentration of 0.2 mol/l. In addition, the solution 2 comprised 10.6 g/L of sodium carbonate (Riedel-de-Haen) and 50 g/l of Sokalan® HP 80 (BASF SE, solids content=40% by weight).

800 ml of the solution 2 were metered at room temperature into 800 ml of the solution 1, with stirring in the course of 12 minutes. The resulting reaction mixture was then stirred for a further 12 minutes. The green suspension obtained was transferred via a 0.45 μm filter. The filtered suspension had a mean particle size of about 65 nm.

### Example 4

Batchwise preparation of nanoparticulate copper hydroxocarbonate in the presence of Sokalan® HP 80 (modified polycarboxylate ether, MW=20 000 g/mol)

Two aqueous solutions 1 and 2 were first prepared. The solution 1 was prepared at room temperature and comprised 49.3 g of copper nitrate (Merck, Cu content 25.8 g/100 g) per liter and had a copper ion concentration of 0.2 mol/l. In addition, the solution 1 comprised 50 g/l of Sokalan® HP 80 (BASF SE, solids content=40% by weight).

The solution 2 comprised 8 g of sodium hydroxide per liter and thus had a hydroxyl ion concentration of 0.2 mol/l. In addition, the solution 2 comprised 10.6 g/L of sodium carbonate (Riedel-de-Haen) and 50 g/l of Sokalan® HP 80 (BASF SE, solids content=40% by weight).

800 ml of the solution 2 were metered at room temperature into 800 ml of the solution 1, with stirring in the course of 12 minutes. The resulting reaction mixture was then stirred for a further 12 minutes. The green suspension obtained was transferred via a 0.45 μm filter. The filtered suspension had a mean particle size of about 83 nm.

1-13. (canceled)

14. A process for the preparation of surface-modified nanoparticulate copper compounds, comprising the steps:
a) preparing an aqueous solution comprising copper ions (solution 1) and an aqueous solution comprising at least one anion which forms a precipitate with copper ions and is not a hydroxide ion (solution 2), at least one of the two solutions 1 and 2 comprising at least one water-soluble polymer,
b) mixing of the solutions 1 and 2 prepared in step a), at a temperature in the range from 0 to 100° C., with the surface-modified nanoparticulate copper compounds forming and being precipitated from the solution with formation of an aqueous dispersion,
c) isolating the surface-modified nanoparticulate copper compounds from the aqueous dispersion obtained in step b), and
d) optionally, drying of the surface-modified nanoparticulate copper compounds obtained in step c).

15. The process according to claim 14, wherein the water-soluble polymer is a polycarboxylate.

16. The process according to claim 14, wherein the concentration of the water-soluble polymer in the solutions 1 and/or 2 prepared in process step a) is in the range from 0.1 to 30 g/l.

17. The process according to claim 14, wherein the particle size of the surface-modified nanoparticulate copper compounds prepared is in the range from 1 to 200 nm.

18. A surface-modified nanoparticulate copper compound having a chemical composition according to the general formula
[Cu²⁺]₁₋ₓ[M^{k+}]ₓ[Xⁿ⁻]ₐ[Y^{m-}]_{b}·e H₂O,
where
M^{k+} is a metal ion having the valency k,
0≦x≦0.5,
Xⁿ⁻ is an anion having the valency n, which forms a precipitate with copper ions and is not a hydroxide ion,
Y^{m-} is an anion having the valency m,
a>0, b≧0 and the ratio of a, b and x is dependent on the valencies k, n and m according to the formula a·n+b·m=2·(1-x)+x·k,
e≧0,
having a particle diameter of from 1 to 200 nm, which copper compound is obtainable by the process according to claim 14.

19. The copper compound according to claim 18, wherein x is 0.

20. The copper compound according to claim 18, wherein Xⁿ⁻ being selected from the group consisting of carbonate, phosphate, hydrogen phosphate, oxalate, borate and tetraborate ions.

21. An antimicrobial active substance or catalyst which comprises a surface-modified nanoparticulate copper compound which is prepared by the process according to claim 14.

22. An antimicrobial active substance or a catalyst which comprises the surface-modified nanoparticulate copper compound according claim 18.

23. A process for the preparation of an aqueous dispersion of a surface-modified nanoparticulate copper compound, comprising the steps
a) preparing an aqueous solution comprising copper ions (solution 1) and an aqueous solution comprising at least one anion which forms a precipitate with copper ions and is not a hydroxide ion (solution 2), at least one of the two solutions 1 and 2 comprising at least one water-soluble polymer,
b) mixing of the solutions 1 and 2 prepared in step a), at a temperature in the range from 0 to 100° C., with the surface-modified nanoparticulate copper compounds forming and being precipitated from the solution with formation of an aqueous dispersion,
c) optionally, concentrating the resulting aqueous dispersion and/or removal of by-products.

24. The process according to claim 23, wherein the water-soluble polymer is a polycarboxylate.

25. An aqueous dispersion of surface-modified nanoparticulate copper compound which has a chemical composition according to the general formula
[Cu²⁺]₁₋ₓ[M^{k+}]ₓ[Xⁿ⁻]ₐ[Y^{m-}]_{b}·e H₂O,
where
M^{k+} is a metal ion having the valency k,
0≦x≦0.5,
Xⁿ⁻ is an anion having the valency n, which forms a precipitate with copper ions and is not a hydroxide ion,
Y^{m-} is an anion having the valency m,
a>0, b≧0 and the ratio of a, b and x is dependent on the valencies k, n and m according to the formula a·n+b·m=2·(1-x)+x·k,
e≧0,
having a particle diameter of from 1 to 200 nm, which dispersion is obtainable by the process according to claim 23.

26. An antimicrobial active substance or a catalyst which comprises the aqueous dispersion of surface-modified nanoparticulate copper compound according to claim 25.
